(19) United States
(12) Patent Application Publication
(10) Pub. No.: US 2022/0293229 A1
(43) Pub. Date: Sep. 15, 2022

(54) DENTAL CARE SERVICE PROVIDING SYSTEM AND DENTAL CARE SERVICE PROVIDING METHOD USING SAME

(71) Applicant: NEWTON1665 CO., LTD., Seoul (KR)

(72) Inventor: Mira PARK, Seoul (KR)

(21) Appl. No.: 17/633,539

(22) PCT Filed: Nov. 17, 2019
(86) PCT No.: PCT/KR2019/015716

§ 371 Date: Feb. 7, 2022

(30) Foreign Application Priority Data
Aug. 5, 2019 (KR) 10-2019-0095242

(51) Int. Cl. G16H 10/60; G06Q 50/22; G16H 30/20

(52) U.S. Cl. CPC G16H 10/60; G06Q 50/22; G16H 30/20

(57) ABSTRACT

The present invention provides a method for providing a dental care service using a server, a user terminal communicating with the server, and a dental cleaner terminal communicating with the server. The method includes performing a dental cleaner matching information transmission operation, in which teeth cleaning service user information is transmitted from the user terminal to the server, performing a dental cleaner list transmission operation, in which a created dental cleaner list is transmitted from the server to the user terminal, and performing a dental cleaner matching request operation, in which dental cleaner information selected from the created dental cleaner list is transmitted from the user terminal to the server.

### BACKGROUND

The present disclosure relates to a system for providing a dental care service and a method for providing a dental care service using the same.

Elderly patients or patients with neural damage, who live in a bed rest state, may be unable to close their mouths by themselves due to the influence of gravity, and thus may frequently suffer from pneumonia that causes even death.

To describe the progress of pneumonia in more detail, when a mouth is kept open and thus becomes dry, symbiotic resident bacteria which are present in a moist oral cavity disappear and pathogenic bacteria which thrive in dry environments sharply increase, and thus oral immunity is rapidly weakened. Thereafter, bacteria that have easily come into the open mouth rapidly reproduce, and pathogenic bacteria may be introduced into the lungs through the airways due to mistaken swallowing that may occur when swallowing muscles are out of balance since the tongue is displaced downwards while the mouth is kept open, thus causing pneumonia. To treat the pneumonia, antibiotics may be repeatedly prescribed but may be no longer effective later due to antibiotic resistance.

Statistically, as of 2016, at least 65 percent of all pneumonia patients are elderly patients aged 60 years and over. According to the statistics of the Korea National Statistical Office, pneumonia is ranked 4th among the top 10 causes of death, and it is confirmed that pneumonia has been ranked up by 6 levels in 10 years.

Meanwhile, when muscles form an open vector system since the upper and lower lips are kept parted, a tensile force, compressive force, and torsional stresses are concentrated on a particular body part, thus causing body imbalance.

That is, anatomically, human body muscles should be stable by forming a closed vector system so that the sum of tensile force, compressive force, and torsional stresses becomes 0 in any moment such as when lying, when sitting, when standing still, when walking, or when running.

However, when the open vector system is continuously formed, chronic pain may occur in a part of a body, or various diseases may occur due to body imbalance such as dental malocclusion, snoring, sleep apnea, rhinitis, facial asymmetry, straight neck syndrome, turtle neck syndrome, spinal disc herniation, strabismus, and the like.

In particular, elderly patients or patients with neural damage may suffer a breathing disorder due to deformation of the maxilla and mandible and the tongue being twisted, or may suffer a serious problem such as spinal or lumbar disc herniation.

As a result, elderly patients or patients with neural damage require a care service such as installation of a mouth closing aid and an oral structure correction treatment for mandibular position recovery. In addition, elderly patients or patients with neural damage require a periodic teeth cleaning service, that is like periodic toothbrushing performed by ordinary people, for removing a bacterial membrane in the mouth and preventing bacteria from being introduced into the mouth so that their oral cleanliness can be maintained.

However, since it is difficult for all medical institutions to have relevant experts, it is difficult for patients with impaired mobility to practically receive such a care.

Therefore, a service for more easily providing the above-mentioned required cares is necessary, and, further, it is necessary to improve the quality of a management service.

### SUMMARY

The present disclosure is intended to allow a patient to easily use a dental care service.

In detail, the present disclosure is intended to improve accessibility of patient with impaired mobility to a teeth cleaning service and a correction treatment service.

The present disclosure is also intended to allow a patient or guardian to confirm, in real time, improvement effects due to a teeth cleaning service and a correction treatment.

In accordance with an exemplary embodiment of the present invention, a method for providing a dental care service using a server, a user terminal communicating with the server, and a dental cleaner terminal communicating with the server includes: performing a dental cleaner matching information transmission operation, in which teeth cleaning service user information is transmitted from the user terminal to the server; performing a dental cleaner list transmission operation, in which a created dental cleaner list is transmitted from the server to the user terminal; and performing a dental cleaner matching request operation, in which dental cleaner information selected from the created dental cleaner list is transmitted from the user terminal to the server.

In an exemplary embodiment, the method may further include: performing a teeth cleaning request operation, in which the teeth cleaning service user information is transmitted from the server to a cleaner terminal corresponding to the dental cleaner information; and performing a teeth cleaning matching decision operation, in which teeth cleaning matching decision information is transmitted from the dental cleaner terminal to the server.

In an exemplary embodiment, the method may further include performing a teeth cleaning guide information transmission operation, in which information of at least one of a teeth cleaning method or precautions when cleaning teeth is transmitted from the server to the dental cleaner terminal.

In an exemplary embodiment, the method may further include performing a before-teeth-cleaning dental condition information transmission operation, in which dental condition information including a teeth image of a user before teeth cleaning is transmitted from the dental cleaner terminal to the server.

In an exemplary embodiment, the method may further include performing an after-teeth-cleaning dental condition information transmission operation, in which dental condition information including a teeth image of the user after teeth cleaning is transmitted from the dental cleaner terminal to the server.

In an exemplary embodiment, the dental cleaner matching request operation may be performed only when it is determined that user's agreement on protection of dental cleaner personal information is completed in the user terminal.

In an exemplary embodiment, the method may further include: performing an image transmission operation, in which a body skeleton image of a user is transmitted from the user terminal to the server; performing a skeleton condition analysis operation, in which skeleton information in which a skeleton balance state is digitized is derived by analyzing the body skeleton image in the server; and performing a skeleton condition information transmission operation, in which the skeleton information is transmitted from the server to the user terminal.

In an exemplary embodiment, the method may further include performing a skeleton condition information usage agreement operation, in which information indicating user's agreement on usage of skeleton condition information of the user for a medical research purpose is transmitted from the user terminal to the server.

In an exemplary embodiment, the method may further use a dentist terminal communicating with the server, and may further include: performing a dentist matching information transmission operation, in which correction service user information is transmitted from the user terminal to the server; performing a dentist list transmission operation, in which a created dentist list is transmitted from the server to the user terminal; and performing a correction treatment matching request operation, in which dentist information selected from the created dentist list is transmitted from the user terminal to the server.

In an exemplary embodiment, the method may further include performing a correction device delivery request operation, in which correction device delivery information is transmitted from the server to the dental cleaner terminal.

### BRIEF DESCRIPTION OF THE DRAWINGS

Exemplary embodiments can be understood in more detail from the following description taken in conjunction with the accompanying drawings, in which:

FIG. 1 is a schematic diagram illustrating a system for implementing a dental care service providing method according to an embodiment of the present invention;

FIG. 2 illustrates a main page provided to a user terminal by a dental care service providing method according to an embodiment of the present invention;

FIG. 3 is a block diagram illustrating a detailed configuration of the system for implementing a dental care service providing method;

FIG. 4 illustrates a dental cleaner matching flow of the dental care service providing method;

FIGS. 5 to 7 illustrate pages that implement the dental cleaner matching flow, which are provided to a user terminal by a dental care service providing method according to an embodiment of the present invention;

FIGS. 8 to 12 illustrate pages that implement the dental cleaner matching flow, which are provided to a dental cleaner terminal by a dental care service providing method according to an embodiment of the present invention;

FIG. 13 illustrates a skeleton condition analysis flow of the dental care service providing method;

FIGS. 14 to 18 illustrate pages that implement the skeleton condition analysis flow, which are provided to a user terminal by a dental care service providing method according to an embodiment of the present invention;

FIG. 19 illustrates a correction service matching flow of the dental care service providing method;

FIGS. 20 to 22 illustrate pages that implement the correction service matching flow, which are provided to a user terminal by a dental care service providing method according to an embodiment of the present invention; and

FIGS. 23 to 25 illustrate pages that implement the correction service matching flow, which are provided to a dentist terminal by a dental care service providing method according to an embodiment of the present invention.

### DETAILED DESCRIPTION OF EMBODIMENTS

Hereinafter, various embodiments of the present invention will be described with reference to the accompanying drawings.

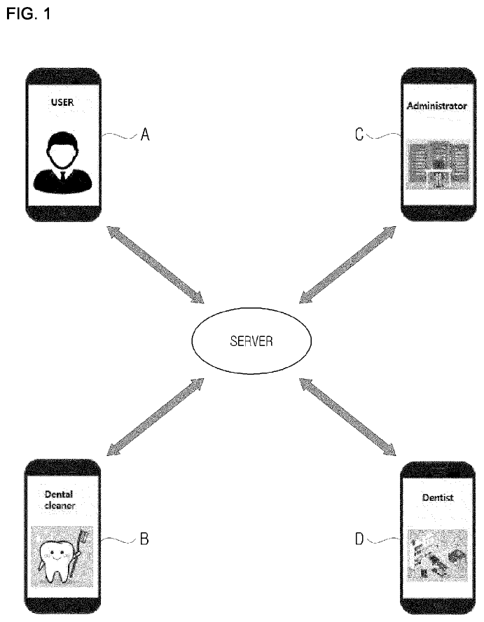

FIG. 1 is a schematic diagram illustrating a dental care service providing system for implementing a dental care service providing method.

The dental care service providing system according to an embodiment of the present invention may include a server S, which is accessible through wired or wireless communication and stores information, a user terminal A, which transmits/receives information to/from the server S through wired or wireless communication, a dental cleaner terminal B, which transmits/receives information to/from the server S through wired or wireless communication, an administrator terminal C, which transmits/receives information to/from the server S through wired or wireless communication, and a dentist terminal D, which transmits/receives information to/from the server S through wired or wireless communication.

Here, the “user” may refer to an individual, a medical institution, or the like, the “administrator” may refer to a medical institution, a government agency, a dental cleaning service provider, or the like, and the “dentist” may refer to a commonly known dentist.

Meanwhile, the “dental cleaner” may be newly defined herein as a person who directly visits a patient and provides a dental care service (toothbrushing, installation of a mouth closing aid, installation of a correction device, confirmation of correction progress, or the like) like periodic toothbrushing performed by ordinary people.

A plurality of the user terminals A, the dental cleaner terminals B, the administrator terminals C, and the dentist terminals D may be connected to the server S as necessary.

Here, the “terminal” may encompass any unit including equipment for transmission through a wired or wireless Internet network.

That is, the “terminal” corresponds to equipment for access through the Internet, and may broadly include an input device such as a keyboard, a mouse, or the like for inputting various data, an output device such as a monitor, a printer, or the like, a transmission/reception device such as a modem, LAN, or the like, a processing device such as a microprocessor or graphics card, and a storage device in which various programs for operating or controlling the foregoing devices are installed.

For example, the “terminal” may include a personal communication system (PCS), a global system for mobile communications (GSM) terminal, a personal digital cellular (PDC) terminal, a personal handyphone system (PHS) terminal, a personal digital assistant (PDA), a smart phone, telematics, a wireless data communication terminal and/or mobile Internet terminal, a personal computer, a notebook, etc.

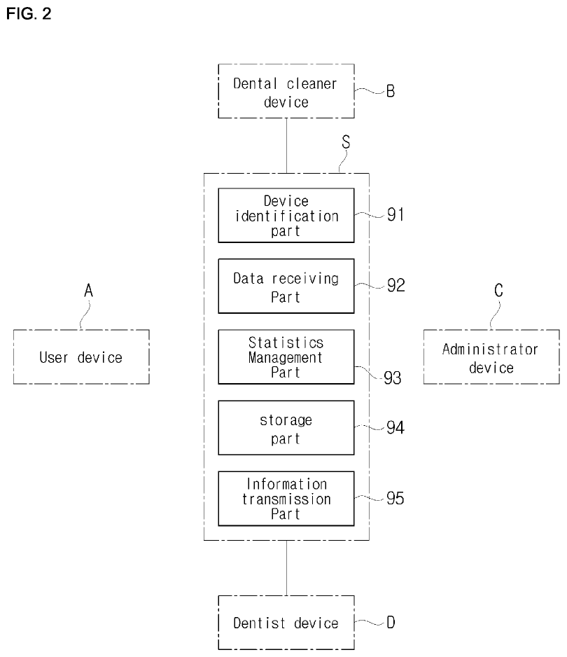

Hereinafter, individual configurations of the server S for implementing the dental care service providing system will be described with reference to FIG. 2.

The server S may include a terminal identification part 91, which identifies the user terminal A, the dental cleaner terminal B, the administrator terminal C, and the dentist terminal D with which the server S is communicating, a data receiving part 92, which receives data from the foregoing terminals, a statistics management part 93, which derives information by processing received information, a storage part 94, which accumulates and stores data and information, and an information transmission part 95, which transmits information to the terminals.

The server S may be configured with a dedicated hardware system and wired/wireless communication network for implementing the dental care service providing method, and may also be implemented through a virtual file-based web cloud service.

The dental care service providing method may be divided into a teeth cleaning service providing flow, a skeleton change analysis flow, and a correction service providing flow.

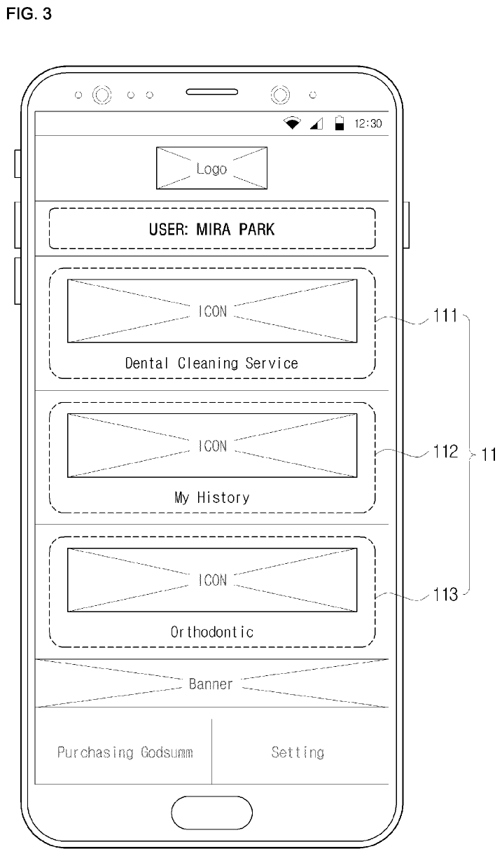

Referring to FIG. 3, a display of the user terminal A may be provided with a main page 11, on which provision services are arranged, and a teeth cleaning service selection area 111 for matching a user and a dental cleaner, a history lookup selection area 112 for looking up a skeleton change history of a user, and a correction service selection area 113 for matching a user (patient) and a dentist may be separately arranged according to the above division.

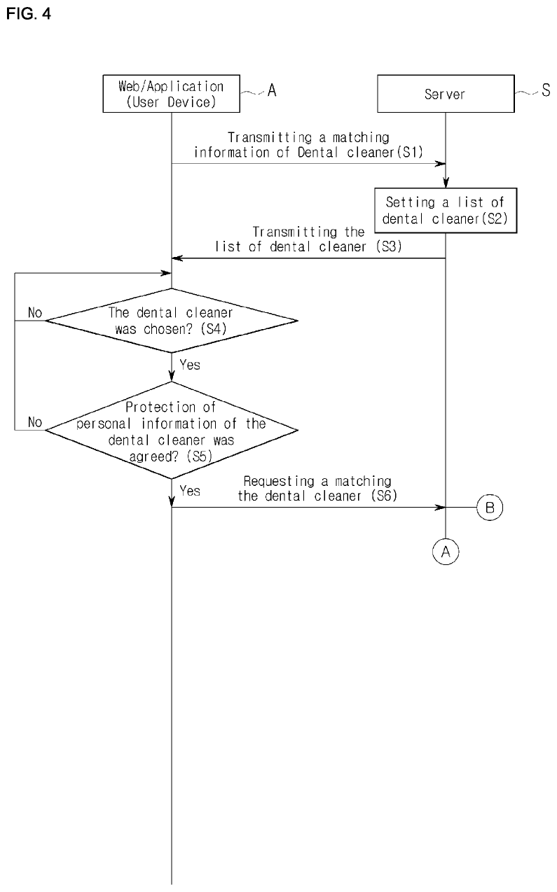

When the teeth cleaning service selection area 111 is selected from the main page 11, the teeth cleaning service flow may progress. The teeth cleaning service flow will be described in detail with reference to FIG. 4.

The dental care service providing method according to an embodiment of the present invention may include a dental cleaner matching information transmission operation S1 in which teeth cleaning service user information is transmitted from the user terminal A to the server S.

The teeth cleaning service user information may include a name/age/gender of the user, a name/phone number of a guardian, a service reservation date/time, and a service location (address).

Accordingly, the user himself/herself may request a teeth cleaning service, and also a guardian (not only a legal guardian but also a medical institution where the user resides is possible) may request the teeth cleaning service on behalf of the user when the user has a serious difficulty in mobility.

Next, the dental care service providing method according to an embodiment of the present invention may further include a dental cleaner list creation operation S2 in which a dental cleaner capable of providing a teeth cleaning service is extracted from the server S.

In the dental cleaner list creation operation S2, dental cleaners who are located within a predetermined distance from a service location input by the user and have no reservation at the service date/time input by the user may be extracted and listed.

Furthermore, listed dental cleaners may be re-sorted in descending order of ratings given by users.

Next, the method may proceed with a dental cleaner list transmission operation S3, in which a list of re-sorted dental cleaners is transmitted from the server S to the user terminal A. Accordingly, the user terminal A may be provided with a list of dental cleaners capable of being requested to be matched.

The dental cleaner list may contain at least one piece of information among a name, gender, career, and rating of each dental cleaner.

Thereafter, when a specific dental cleaner is selected by the user, the method may proceed with a dental cleaner selection determination operation S4 in the user terminal A.

Accordingly, the user or guardian may request a teeth cleaning service from a desired dental cleaner among the listed dental cleaners, and thus the user may easily use the service even when the user stays in a medical institution or family house where a professional dental cleaner does not reside.

Next, when a specific dental cleaner is determined to be selected in the dental cleaner selection determination operation S4, the method may proceed with an agreement-to-protection-of-dental-cleaner-personal-information determination operation S5 in the user terminal A.

The agreement-to-protection-of-dental-cleaner-personal-information determination operation S5 is a procedure for protecting privacy of a matched dental cleaner by preventing the user or guardian from leaking personal information of the dental cleaner.

When it is determined that agreement is not selected (disagreement) in the agreement-to-protection-of-dental-cleaner-personal-information determination operation S5, the dental cleaner list may be re-created, and the dental cleaner selection determination operation S4 may be performed again.

On the contrary, when agreement is selected in the agreement-to-protection-of-dental-cleaner-personal-information determination operation S5, the method may sequentially proceed with a dental cleaner matching request operation S6, in which dental cleaner information selected from the created dental cleaner list is transmitted from the user terminal A to the server A, and a teeth cleaning request operation S7, in which the teeth cleaning service user information is transmitted from the server S to the dental cleaner terminal B of a dental cleaner corresponding to the dental cleaner information.

Thereafter, in the dental cleaner terminal B, the method may proceed with a teeth cleaning request approval determination operation S8, in which it is identified whether the dental cleaner has given a matching approval to the teeth cleaning request of the user.

The teeth cleaning request approval determination operation S8 exhibits an effect in which a dental cleaner may also choose whether to accept a user's teeth cleaning request and thus may flexibly adjust a personal schedule.

When the teeth cleaning request is determined not to be approved in the teeth cleaning request approval determination operation S8, the matching procedure may be interrupted, and a matching rejection message may be provided through the user terminal A, and, when the teeth cleaning request is determined to be approved, the method may proceed with an agreement-to-protection-of-user-personal-information determination operation S9 in the dental cleaner terminal B.

The agreement-to-protection-of-user-personal-information determination operation S9 is also a procedure for protecting privacy of a matched user by preventing the dental cleaner from leaking personal information of the user.

When it is determined that agreement is not selected (disagreement) in the agreement-to-protection-of-user-personal-information determination operation S9, the matching procedure may be interrupted, and a matching rejection message may be provided through the user terminal A.

On the contrary, when it is determined that agreement is selected in the agreement-to-protection-of-user-personal-information determination operation S9, the method may proceed with a teeth cleaning matching decision operation S10, in which teeth cleaning matching decision information is transmitted from the dental cleaner terminal B to the server S, and detailed information (detailed address, dental condition information, oral condition information, etc.) of the user may be transmitted from the server S to the dental cleaner terminal B.

According to the above-mentioned flow, a user and a dental cleaner may be flexibly matched, and, during this process, leakage of sensitive medical and job information may also be prevented by carrying out a procedure for protecting personal information.

Even when a dental cleaner is matched, it is necessary to maintain quality of a teeth cleaning service so that a service difference between dental cleaners does not significant.

Therefore, the dental care service providing method according to an embodiment of the present invention may further include, as a technical configuration for service quality management, a teeth cleaning guide information transmission operation S11, in which teeth cleaning guide information is transmitted from the server S to the dental cleaner terminal B.

The teeth cleaning guide information may include at least one of a teeth cleaning method or precautions when cleaning teeth, wherein the precautions may be related to use of single-use equipment and mandatory wearing of a protective gear for a dental cleaner.

Thereafter, when it is determined that the precautions have been confirmed by the dental cleaner in the dental cleaner terminal B, the method may proceed with a before-teeth-cleaning dental condition information transmission operation S12, in which dental condition information of the user before teeth cleaning is transmitted from the dental cleaner terminal B to the server S.

The dental condition information of the user before teeth cleaning may include a teeth image of the user, wherein the teeth image may be an image in which a medical coloring agent is applied to teeth so that adherent bacteria may be visually identified, and, in addition, a facial skeleton image of the user may be added to the teeth image.

Thereafter, the method may proceed with an after-teeth-cleaning dental condition information transmission operation S13, in which dental condition information of the user after teeth cleaning is transmitted from the dental cleaner terminal B to the server S.

The dental condition information of the user after teeth cleaning may include a teeth image of the user in which the medical coloring agent has been removed, and may further include an image in which a mouth closing aid is attached to a face of the user.

Here, the above-mentioned dental condition information of the user before/after teeth cleaning may be looked up through the user terminal A.

According to the above technical configuration, an overall process of the teeth cleaning service may be monitored by the user, guardian, and administrator, and thus service quality may be maintained at a high level.

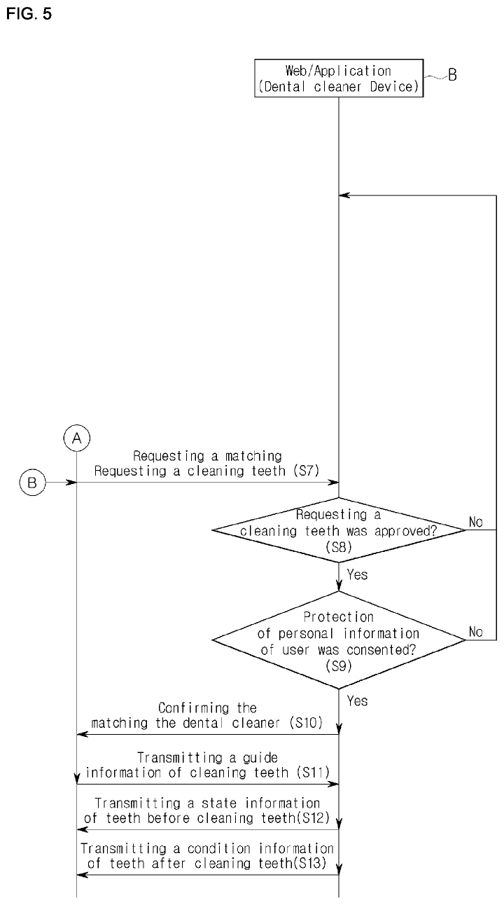

FIGS. 5 to 7 illustrate an example in which the teeth cleaning service providing flow that is a part of the dental care service providing method is implemented through a web/application of the user terminal A.

FIG. 5 illustrates that a teeth cleaning usage information input page 12 is provided through the display of the user terminal A, wherein the teeth cleaning usage information input page 12 is a screen switched from the main page 11 when the teeth cleaning service selection area 111 is selected.

In order for the teeth cleaning service user information to be input, a teeth cleaning user personal information input area 121 for inputting a name/age/gender of the user, a teeth cleaning guardian personal information input area 122 for inputting a name/phone number of the guardian, a reservation time input area 123 for inputting a teeth cleaning service reservation data/time, an address input area 124 for inputting an address of a location where a teeth cleaning service is to be provided, and a teeth cleaning usage information confirmation area 125 may be separately arranged on the teeth cleaning usage information input page 12.

The teeth cleaning service user information input to the teeth cleaning usage information input page 12 may be transferred to the server S through the above-mentioned dental cleaner matching information transmission operation S1.

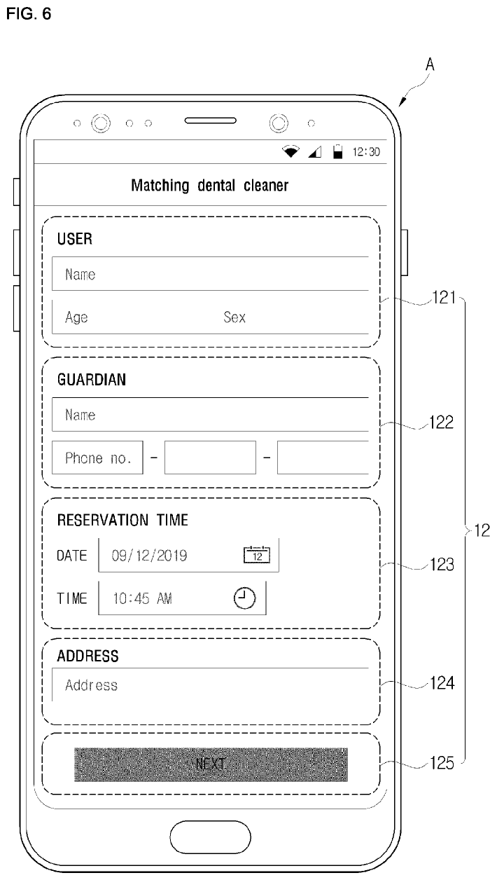

FIG. 6 illustrates a dental cleaner selection page 13, which is switched when the teeth cleaning usage information confirmation area 125 is selected, and on which a dental cleaner list is created and provided.

On the dental cleaner selection page 13, a dental cleaner information area 131, in which pieces of information of dental cleaners such as name/career/rating/review are provided, and a matching request area 132 for selecting a dental cleaner to be requested to be matched may be separately arranged.

When a specific dental cleaner is selected through the matching request area 132, the agreement-to-protection-of-dental-cleaner-personal-information determination operation S5 may be performed according to a result of the dental cleaner selection determination operation S4.

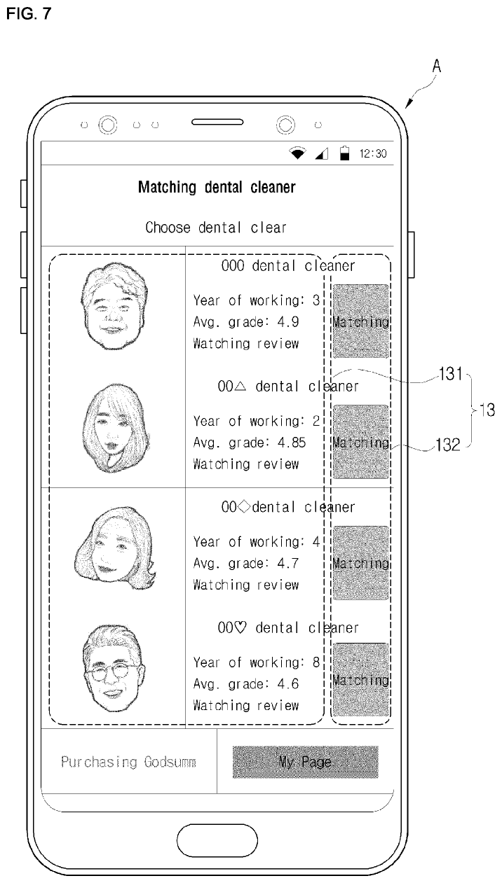

Thereafter, the dental cleaner personal information protection page 14 illustrated in FIG. 7 may be provided to the display of the user terminal A, and a dental cleaner personal information protection explanation area 141, an agreement selection area 142, and a disagreement selection area 143 may be separately arranged on the dental cleaner personal information protection page 14.

The agreement-to-protection-of-dental-cleaner-personal-information determination operation S5 may be performed through the dental cleaner personal information protection page 14.

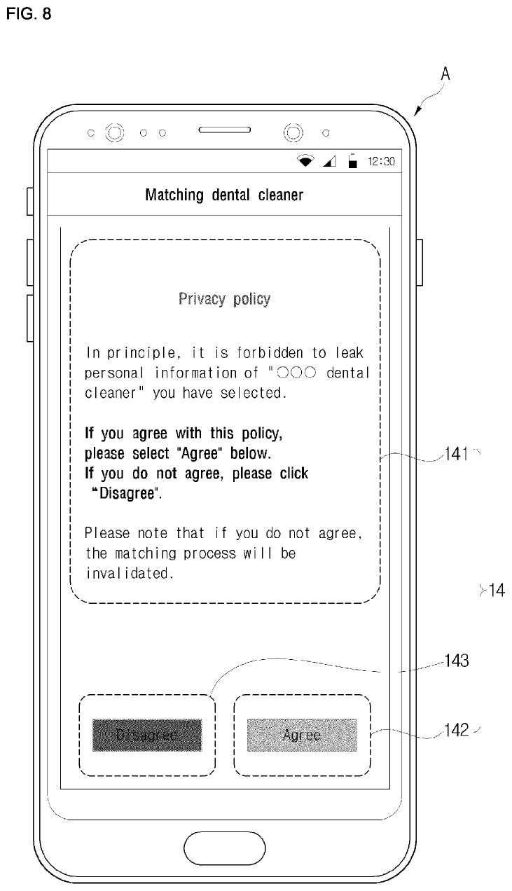

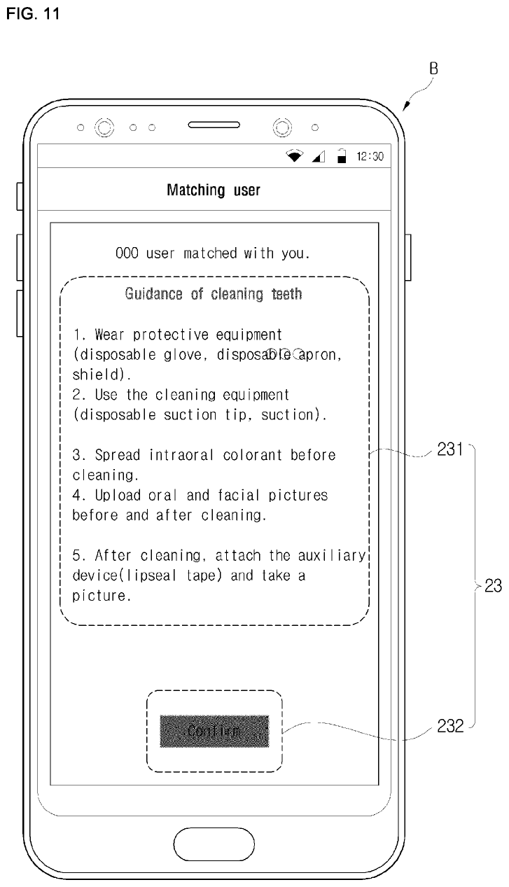

FIGS. 8 to 12 illustrate an example in which the teeth cleaning service providing flow of the dental care service providing method is implemented through a web/application of the dental cleaner terminal B.

Referring to FIG. 8, a teeth cleaning matching page 21 may be provided to a display of the dental cleaner terminal B according to the teeth cleaning request operation S7.

On the teeth cleaning matching page 21, a user information area 211, in which a name/address/reservation time/age/gender of the user are described, a matching approval area 212, and a matching skip area 213 may be separately arranged.

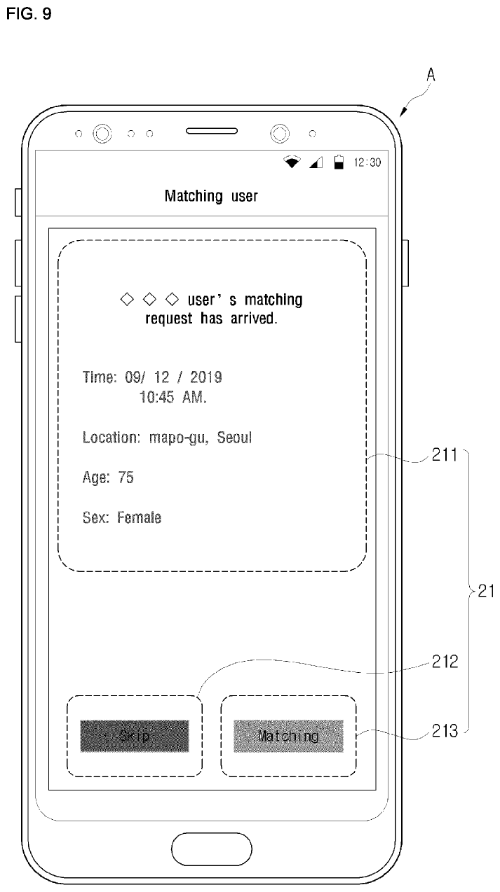

Here, matching may be rejected according to the teeth cleaning request approval determination operation S8 when the matching skip area 213 is determined to be selected, and the page may be switched to the user personal information protection page 22 of FIG. 9 when the matching approval area 212 is determined to be selected.

On the user personal information protection page 22, a user personal information protection explanation area 221, an agreement selection area 222, and a disagreement selection area 223 may be separately arranged.

The agreement-to-protection-of-user-personal-information determination operation S9 may be performed through the user personal information protection page 22.

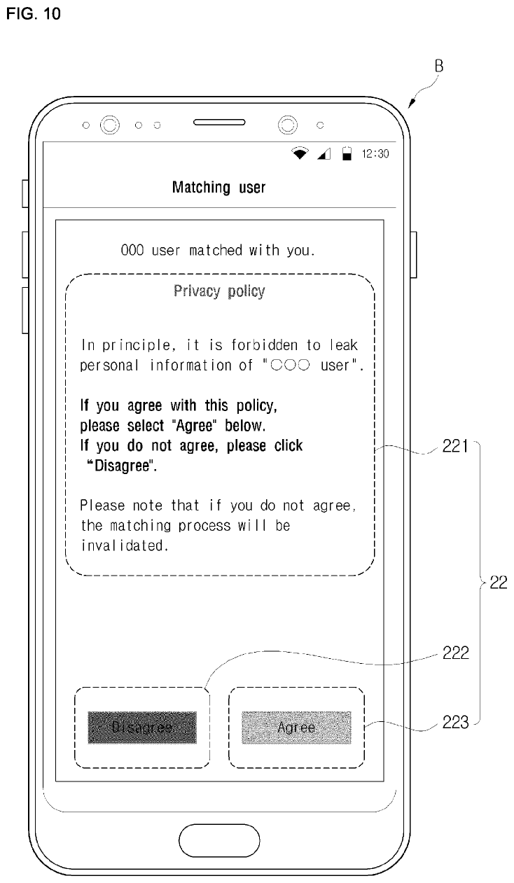

FIG. 10 illustrates a teeth guide page 23 provided through the teeth cleaning guide information transmission operation S11, wherein a teeth cleaning explanation area 231, in which the above-mentioned teeth cleaning method and precautions when cleaning teeth are described, and a confirmation area 232 may be separately arranged.

When the confirmation area 232 is selected from the teeth guide page 23, a before-teeth-cleaning photo page 24 may be provided to the display of the dental cleaner terminal B.

On the before-teeth-cleaning photo page 24, a before-teeth-cleaning photo upload area 241 for uploading images of teeth and a facial skeleton of the user, which are ones of pieces of dental condition information of the user, and a before-teeth-cleaning photo upload confirmation area 242 may be separately arranged.

As described above, in the before-teeth-cleaning photo upload area 241, images showing teeth of the user to which a medical coloring agent (which is discolored during bacteria reaction) is applied may be uploaded.

Furthermore, in the before-teeth-cleaning photo upload area 241, a facial skeleton image of the user may be uploaded. The above-mentioned images may be captured along a guide line (dashed line) and uploaded.

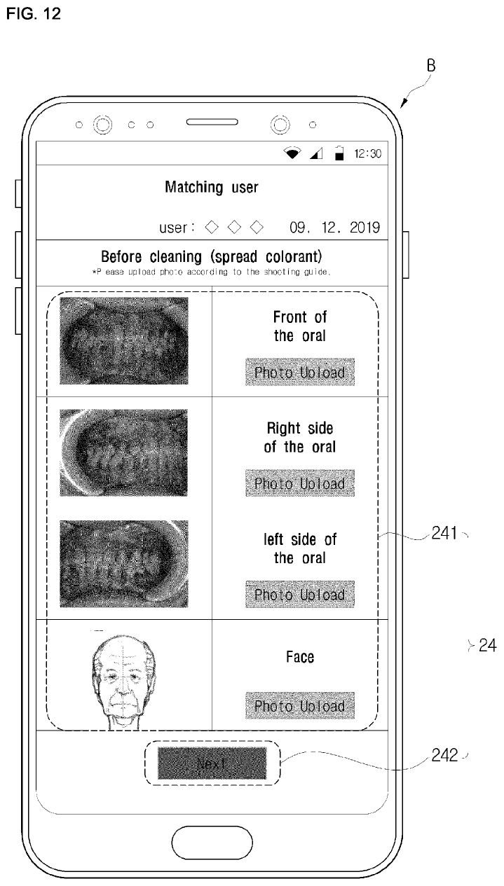

When the before-teeth-cleaning photo upload confirmation area 242 is selected after image uploading, the before-teeth-cleaning dental condition information transmission operation S12 may be performed, and the after-teeth-cleaning photo page 25 illustrated in FIG. 12 may be provided to the display of the dental cleaner terminal B.

Likewise, on the after-teeth-cleaning photo page 25, an after-teeth-cleaning photo upload area 251 for uploading images of teeth and a facial skeleton of the user, which are ones of pieces of dental condition information of the user, and an after-teeth-cleaning photo upload confirmation area 252 may be separately arranged.

In the after-teeth-cleaning photo upload area 251, images showing teeth of the user from which a medical coloring agent (which is discolored during bacteria reaction) has been removed through teeth cleaning may be uploaded.

Meanwhile, in the after-teeth-cleaning photo upload area 251, a facial skeleton image of the user in which a mouth closing aid L is attached across an upper lip and lower lip of the user may be uploaded.

The mouth closing aid L may prevent pneumonia by maintaining a mouth of the user in a closed state, and may prevent a skeleton of the user from being deformed and make the skeleton balanced normally by forming a closed vector system so that a vector sum of muscles near lips becomes 0.

The above-mentioned after-teeth-cleaning images may also be captured along a guide line (dashed line) and uploaded.

Thereafter, when the after-teeth-cleaning photo upload confirmation area 252 is selected, the after-teeth-cleaning dental condition information transmission operation S13 may be performed, and the teeth cleaning service may be finished.

Meanwhile, it is necessary to allow condition information accumulated during the teeth cleaning service to be used by users, guardians, dental cleaners, administrators, and dentists so as to provide a better dental care service to users.

Furthermore, since a procedure for correcting a skeleton through the teeth cleaning service requires a long period of time (six months to three years), it is also necessary to immediately digitize and monitor the procedure so that the user may recognize a correction effect and continuously follow the procedure.

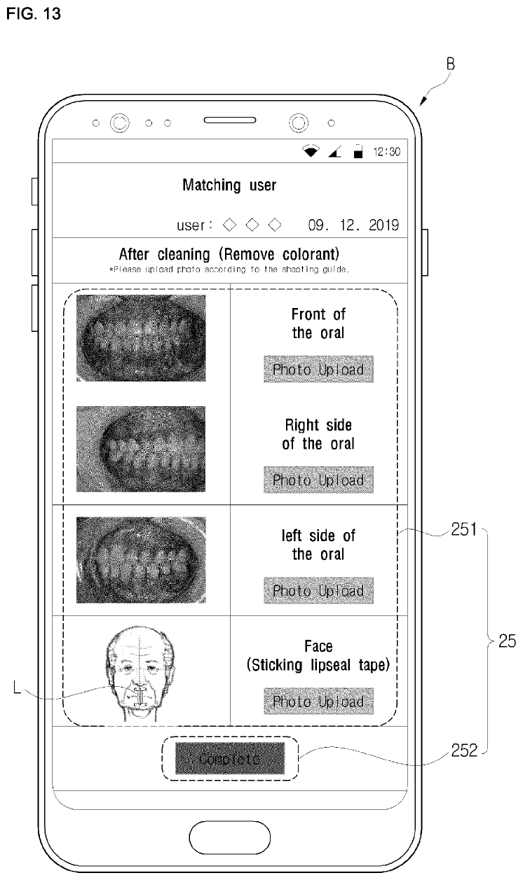

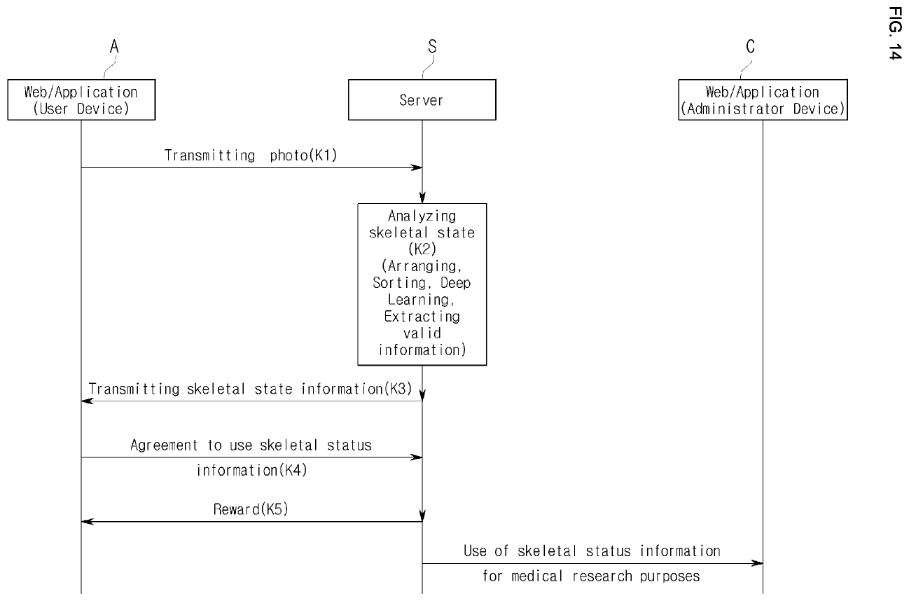

To meet such necessities, the skeleton change analysis flow for analyzing and providing a condition of a skeleton to the user will be described with reference to FIG. 13.

The skeleton change analysis flow may include an image transmission operation K1, in which a body skeleton image is transmitted from the user terminal A to the server S, a skeleton data analysis operation K2, in which the statistics management part 93 of the server S analyzes the body skeleton image to derive skeleton condition information in which a skeleton balance state is digitized, and a skeleton condition information transmission operation K3, in which the skeleton condition information is transmitted from the server S to the user terminal A.

The skeleton condition information may include information about degrees of uniform arrangement and horizontal balance of teeth, a degree of oral horizontal balance, a degree of facial protrusion, and a degree of body balance formation with respect to an inclination of a center axis penetrating the body in a longitudinal direction.

Accordingly, a remarkable effect may be achieved, in which once the user captures images of teeth arrangement, oral cavity, facial skeleton, waist, legs, and full body of the user and uploads the images through a personal terminal, the user may be provided with skeleton information and may also recognize a progress of change according to correction.

Furthermore, the skeleton change analysis flow may include a skeleton condition information usage agreement operation K4, in which information indicating user's agreement on usage of body skeleton information of the user for a medical research purpose is transmitted from the user terminal A to the server S, and a reward operation K5, in which a reward (point, voucher, or the like) is transmitted from the server S to the user terminal A as a benefit in return for the usage agreement.

Accordingly, administrators may use massive big data related to body condition information of users in medical researches.

When the history lookup selection area 112 is selected from the main page 2, the body part selection page 31 illustrated in FIG. 14 may be provided to the display of the user terminal A as an example of specific implementation of the skeleton change analysis flow.

On the body part selection page 31, which is for selecting a body part of which skeleton condition information is to be looked up, a teeth arrangement selection area 311 which is switched to a teeth arrangement analysis page 32 according to selection, an oral cavity selection area 312 which is switched to an oral analysis page 33, a facial skeleton selection area 313 which is switched to a facial skeleton analysis page 34, and a full body skeleton selection area 314 which is switched to a full body skeleton analysis page 35 may be separately arranged.

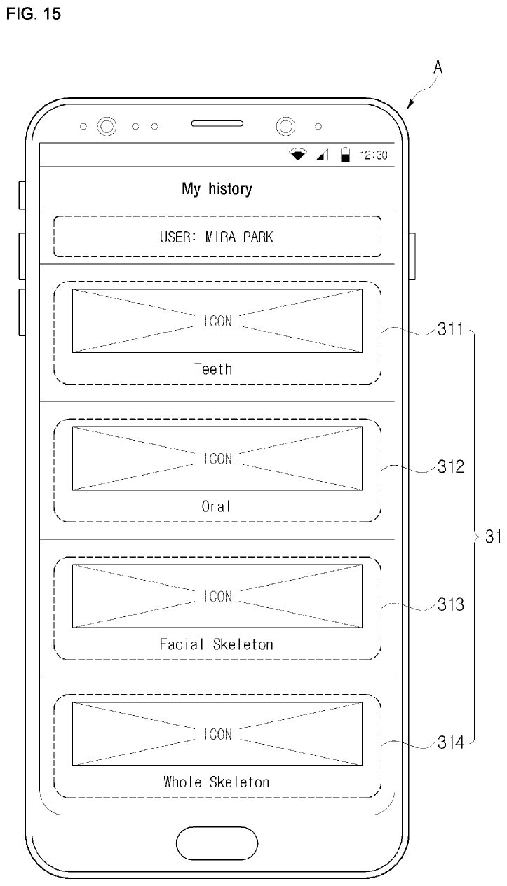

FIG. 15 illustrates the teeth arrangement analysis page 32, on which a teeth arrangement analysis area 321 in which horizontal balance and uniformity of teeth as viewed in front of the teeth are digitized and provided and a teeth arrangement analysis information contribution area 322 for agreeing on usage of teeth arrangement analysis information for a medical research purpose may be separately arranged.

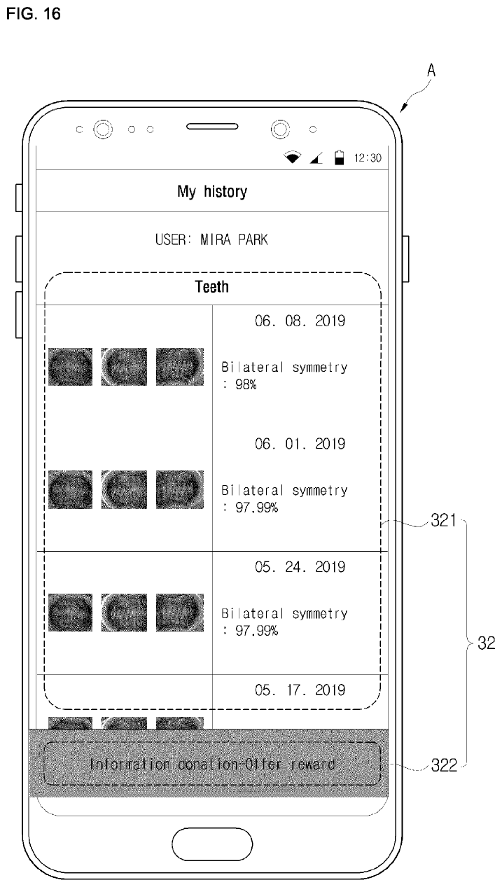

FIG. 16 illustrates the oral analysis page 33, on which an oral analysis area 331 in which a horizontal balance of an oral cavity is digitized and provided and an oral analysis information contribution area 332 for agreeing on usage of oral analysis information for a medical research purpose may be separately arranged.

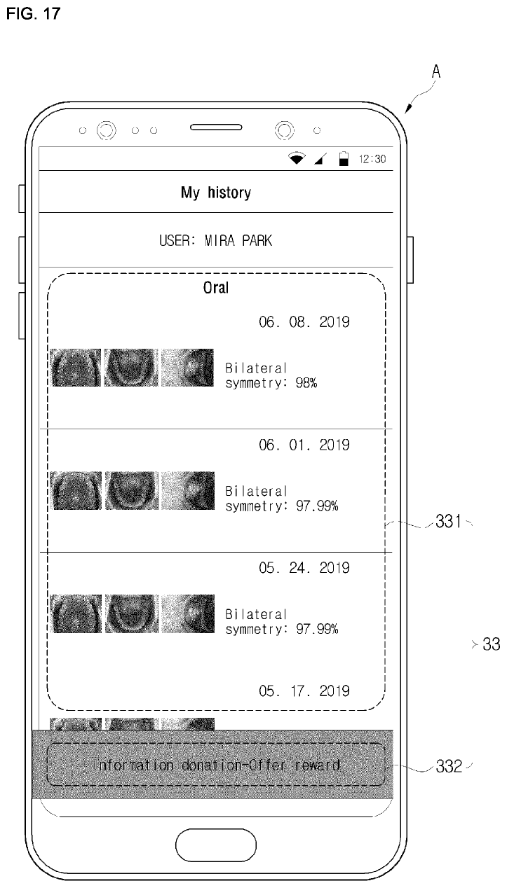

Furthermore, FIG. 17 illustrates the facial skeleton analysis page 34, on which a facial skeleton analysis area 341 in which a horizontal balance and protrusion state of a face are digitized and provided and a facial skeleton analysis information contribution area 342 for agreeing on usage of facial skeleton analysis information for a medical research purpose may be separately arranged.

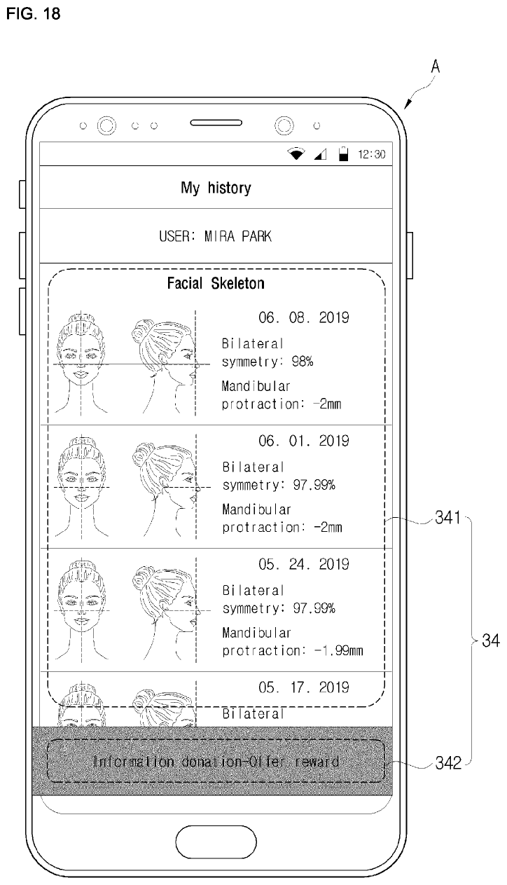

FIG. 18 illustrates the full body skeleton analysis page 35, on which a full body skeleton analysis area 351 in which a body balance related to the inclination of the center axis penetrating the body in a longitudinal direction is digitized and provided and a full body skeleton analysis information contribution area 352 for agreeing on usage of full body skeleton analysis information for a medical research purpose may be separately arranged.

Lastly, the correction service providing flow of the dental care service providing method will be described.

In the present embodiment, the “correction” may include not only a procedure for restoring deformed teeth and skeleton to a normal condition but also a procedure for preventing normal teeth and skeleton structure from being deformed.

There is a mandibular restoration splint device treatment as an example of a correction treatment. This treatment is one of correction treatment methods for securing a lower airway by restoring a mandible displaced backward and downward to a normal position or by preventing further deformation.

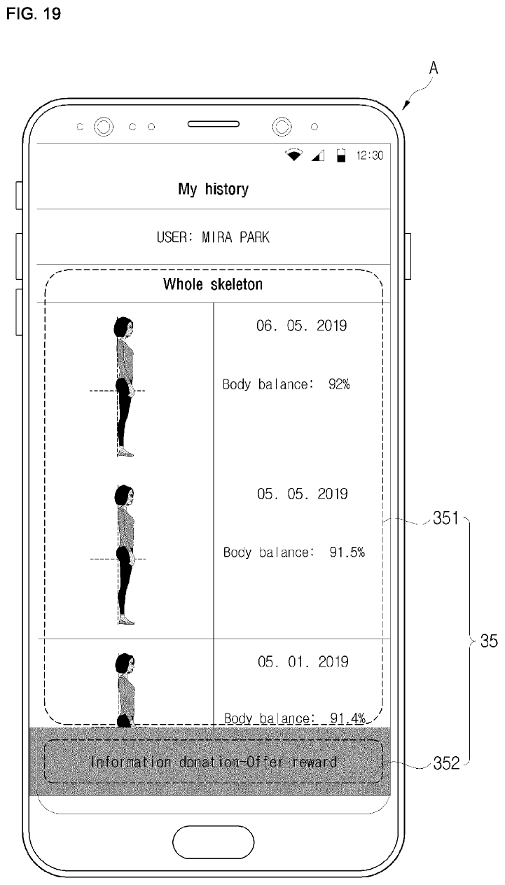

When the correction service selection area 113 is selected from the main page 11, the correction service providing flow may progress. The correction service providing flow will be described in detail with reference to FIG. 19.

The dental care service providing method according to an embodiment of the present invention may include a dentist matching information transmission operation T1 in which correction service user information is transmitted from the user terminal A to the server S.

The correction service user information may include a name/age/gender of the user and a name/phone number, address, and medical insurance number of a guardian.

Therefore, the user (patient) himself/herself may request a correction service, and also a guardian (not only a legal guardian but also a medical institution where the user resides is possible) may request the correction service on behalf of the user when the user has a serious difficulty in mobility.

Next, the dental care service providing method according to an embodiment of the present invention may further include a dentist list creation operation T2, in which a dentist capable of providing a correction service is extracted from the server S.

In the dentist list creation operation T2, dentists who are located within a predetermined distance from an address of the user may be extracted and listed, and listed dentists may be re-sorted in descending order of ratings given by users.

Next, the method may proceed with a dentist list transmission operation T3, in which a list of re-sorted dentists is transmitted from the server S to the user terminal A. Accordingly, the user terminal A may be provided with a list of dentists capable of being requested to be matched.

The dentist list may contain at least one piece of information among a business name, location, and rating of each dentist.

Thereafter, when a specific dentist is selected by the user, the method may proceed with a dentist selection determination operation T4 in the user terminal A.

Accordingly, the user or guardian may request a correction service from a desired dentist among the listed dentists, thus enabling a remote correction treatment.

Next, when a specific dentist is determined to be selected in the dentist selection determination operation T4, the method may proceed with an agreement-to-provision-of-personal-information determination operation T5 in the user terminal A.

The agreement-to-provision-of-personal-information determination operation T5 is a procedure for allowing a dentist to look up personal body condition information (skeleton, teeth cleaning history, correction history) of the user stored in the server S to perform a remote treatment.

When it is determined that agreement is not selected (disagreement) in the agreement-to-provision-of-personal-information determination operation T5, the dentist list may be re-created, and the dentist selection determination operation T4 may be performed again.

On the contrary, when agreement is selected in the agreement-to-provision-of-personal-information determination operation T5, the method may sequentially proceed with a correction treatment matching request operation T6, in which dentist information in the created dentist list is transmitted from the user terminal A to the server S, and a correction treatment request operation T7, in which the correction service user information is transmitted from the server S to the dentist terminal D of a dentist corresponding to the dentist information.

Thereafter, in the dentist terminal D, the method may proceed with a treatment request approval determination operation T8, in which it is identified whether the dentist has given an approval to the correction treatment request of the user.

The treatment request approval determination operation T8 exhibits an effect in which the dentist may also choose whether to treat the user.

When it is determined that approval is not given in the treatment request approval determination operation T8, the matching procedure may be interrupted, and a matching rejection message may be provided through the user terminal.

On the contrary, when it is determined that approval is given in the treatment request approval determination operation T8, the method may proceed with a correction treatment matching decision operation T9, in which correction treatment matching decision information is transmitted from the dentist terminal D to the server.

Thereafter, the method may proceed with a user body condition information transfer operation T10, in which the personal body condition information (skeleton, teeth cleaning history, correction history) of the user is transmitted from the server S to the dentist terminal B.

According to the above-mentioned flow, a user and a dentist may be flexibly matched, and, furthermore, a more efficient remote treatment may be performed using existing accumulated personal data.

Furthermore, when it is necessary to install a correction device according to a result of a correction treatment, the dentist may deliver the correction device to the user (patient) through a dental cleaner and may monitor a progress.

To this end, the dental care service providing method according to an embodiment of the present invention may further include a correction device manufacturing completion information transfer operation T11, in which the dentist terminal D notifies the server S of completion of manufacturing of the correction device, and a correction device delivery request operation T12, in which delivery information including a location (dental laboratory) of a completed correction device and user address information is transmitted from the server S to the dental cleaner terminal B.

Therefore, the correction device may be quickly delivered to the user, and a correction progress may be continuously monitored on the basis of images uploaded through the dental cleaner terminal B.

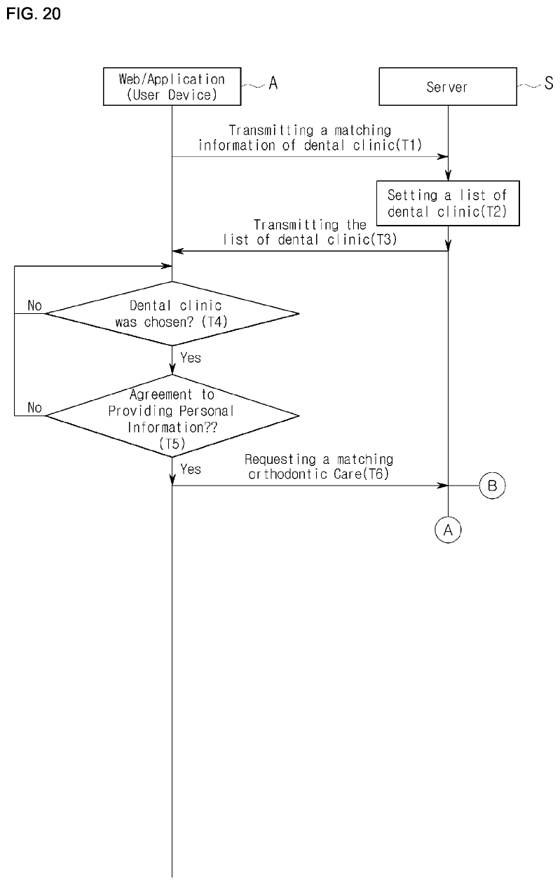

FIGS. 20 to 22 illustrate an example in which the correction service providing flow is implemented through a web/application of the user terminal A.

FIG. 20 illustrates that a correction usage information input page 41 is provided through the display of the user terminal A, wherein the correction usage information input page 12 is a screen switched from the main page 11 when the correction service selection area 113 is selected.

In order for the correction service user information to be input, a correction user personal information input area 411 for inputting a name/age/gender of the user, a correction guardian personal information input area 412 for inputting a name/phone number of the guardian, a medical insurance number input area 413, an address input area 414 for inputting an address of a location of the correction service user, and a correction usage information confirmation area 415 may be separately arranged on the correction usage information input page 41.

The correction service user information input to the correction usage information input page 41 may be transferred to the server S through the above-mentioned dentist matching information transmission operation T1.

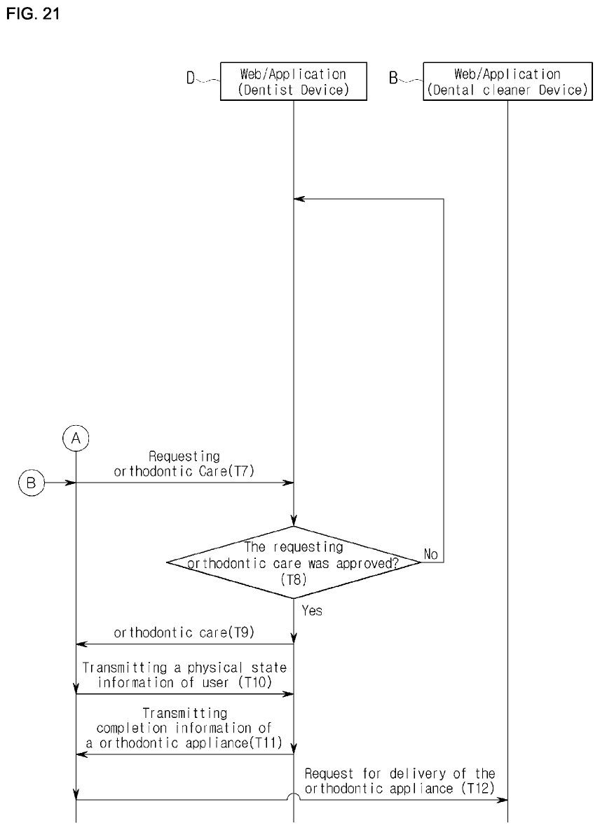

FIG. 21 illustrates a dentist selection page 42, which is switched when the correction usage information confirmation area 414 is selected, and on which a dentist list is created and provided.

On the dentist selection page 42, a dentist information area 421, in which pieces of information of dentists such as business name/rating/address/review are provided, and a treatment request area 422 for selecting a dentist to be requested to treat may be separately arranged.

When a specific dentist is selected through the treatment request area 422, the agreement-to-provision-of-personal-information determination operation T5 may be performed according to a result of the dentist selection determination operation T4.

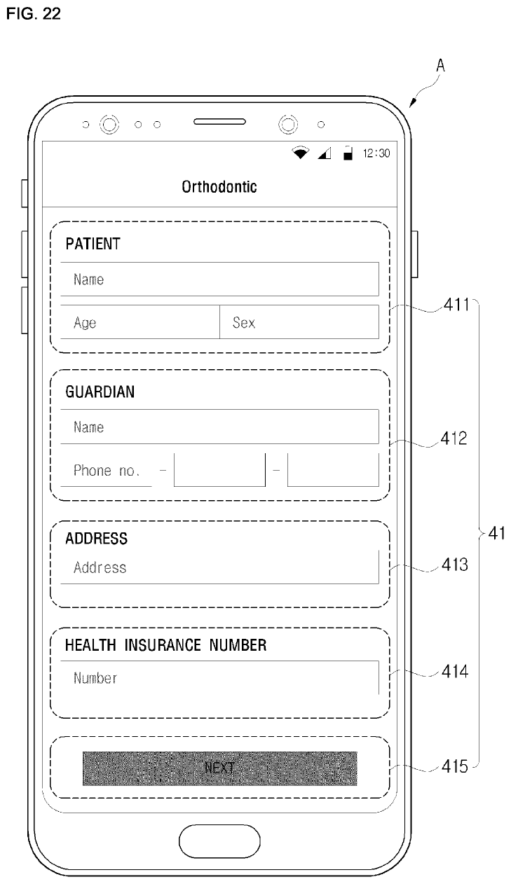

Thereafter, the personal information provision agreement page 43 illustrated in FIG. 22 may be provided to the display of the user terminal A, and a personal information provision explanation area 431, an agreement selection area 432, and a disagreement selection area 433 may be separately arranged on the personal information provision agreement page 43.

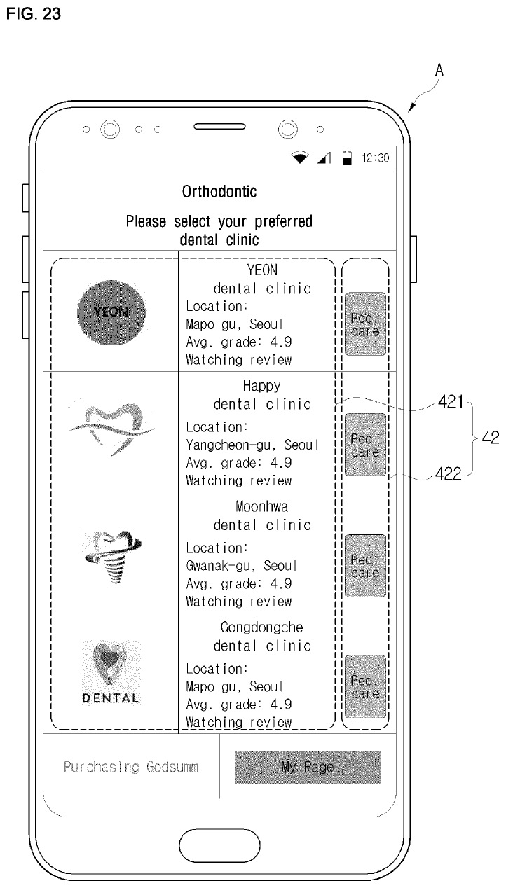

FIGS. 23 to 25 illustrate an example in which the correction service providing flow is implemented through a web/application of the dentist terminal D.

Referring to FIG. 22, a correction treatment approval page 51 may be provided to the dentist terminal D according to the correction treatment request operation T7.

On the correction treatment approval page 51, a user information area 511, in which a name/address/gender of the user are described, a treatment approval area 512, and a treatment skip area 513 may be separately arranged.

Here, matching may be rejected according to the treatment request approval determination operation T8 when the treatment skip area 513 is determined to be selected, and the correction treatment matching decision operation T9 may be performed when the treatment approval area 512 is determined to be selected.

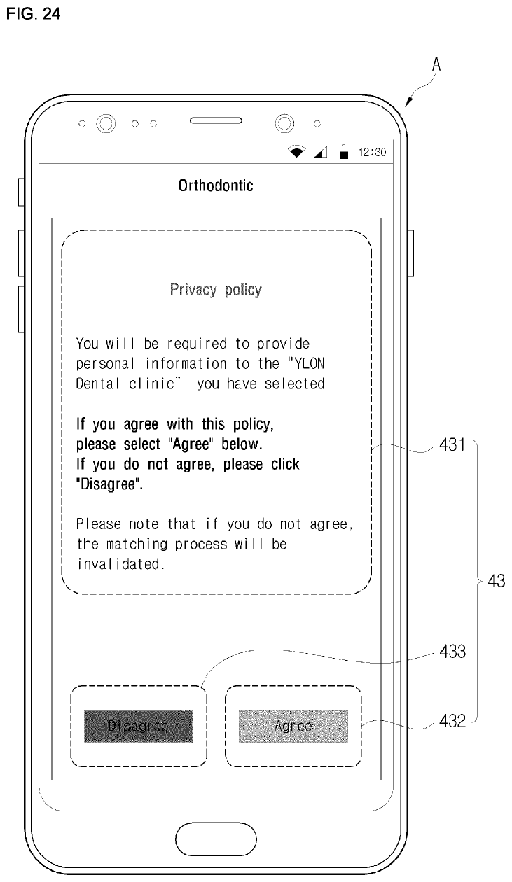

FIG. 24 illustrates a device delivery request page 52 for performing the correction device manufacturing completion information transfer operation T11. On the device delivery request page 52, a delivery information area 521, in which delivery information including at least one of a location (dental laboratory) of a completed correction device, user address information, or a correction device unique code number is described, and a delivery request area 522 may be separately arranged.

Thereafter, when the delivery request area 522 is selected, the method may sequentially proceed with the correction device manufacturing completion information transfer operation T11 and the correction device delivery request operation T12.

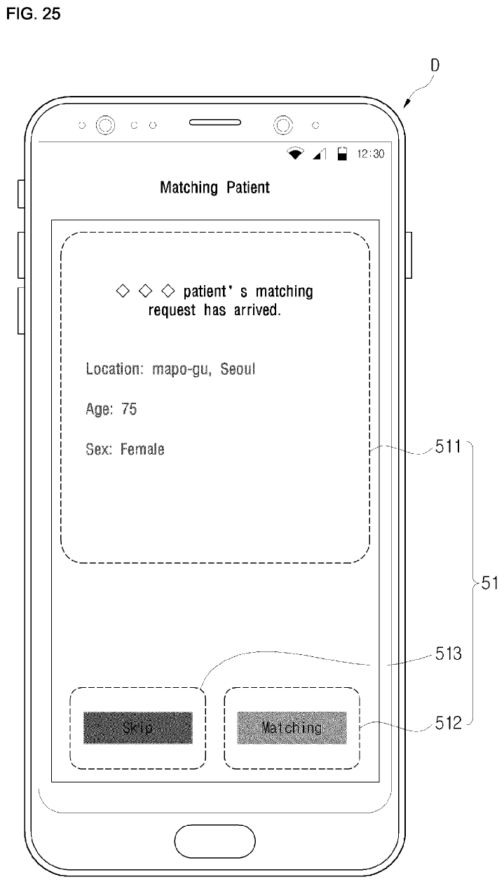

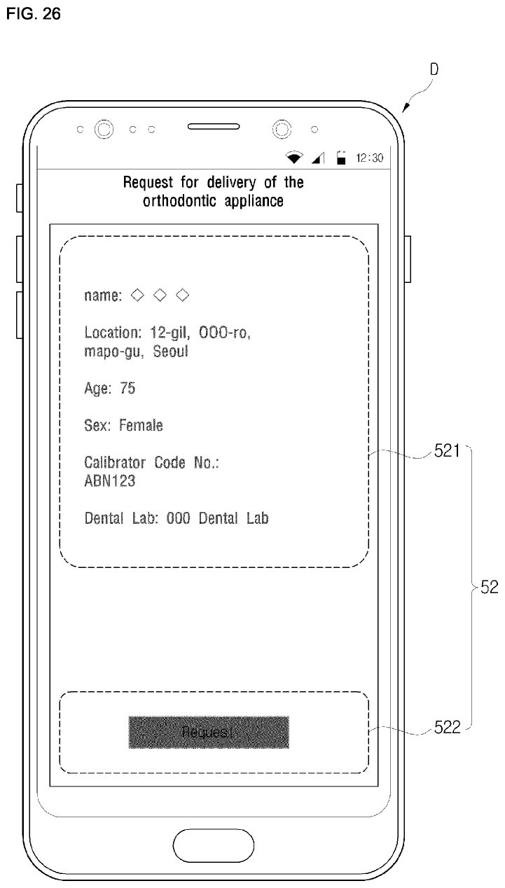

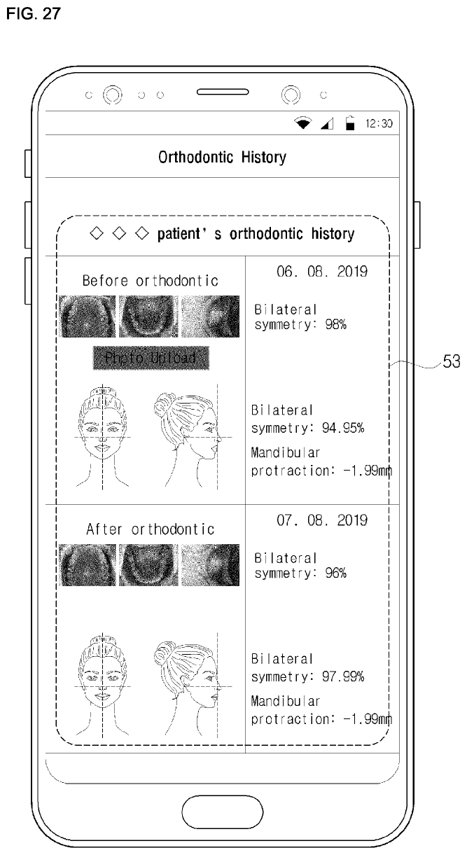

FIG. 25 illustrates a correction history page 53 provided to the user terminal A and the dentist terminal D, as an example in which a correction progress is digitized and provided on the basis of images cumulatively uploaded by the dental cleaner or the user.

According to the present configuration, the user and the dentist may thoroughly check a past correction result and a current correction state.

According to the present invention, a patient may easily use a dental care service.

In detail, according to the present invention, accessibility of patient with impaired mobility to a teeth cleaning service and a correction treatment service may be improved.

Furthermore, according to the present invention, a patient or guardian may confirm, in real time, improvement effects due to a teeth cleaning service and a correction treatment.

Effects as not described herein may be additionally derived from the above configurations. The combinational relationship between the above-described configurations may make it possible to derive new effects over the prior art.

In addition, the embodiments shown in the drawings may be modified and implemented in other forms. The modifications should be regarded as falling within the scope of the present invention when the modifications are carried out so as to include a configuration claimed in the claims or within the scope of an equivalent thereto.

What is claimed is:

1. A method for providing a dental care service using a server, a user terminal communicating with the server, and a dental cleaner terminal communicating with the server, the method comprising:
performing a dental cleaner matching information transmission operation, in which teeth cleaning service user information is transmitted from the user terminal to the server;
performing a dental cleaner list transmission operation, in which a created dental cleaner list is transmitted from the server to the user terminal; and
performing a dental cleaner matching request operation, in which dental cleaner information selected from the created dental cleaner list is transmitted from the user terminal to the server.

2. The method of claim 1, further comprising:
performing a teeth cleaning request operation, in which the teeth cleaning service user information is transmitted from the server to a cleaner terminal corresponding to the dental cleaner information; and
performing a teeth cleaning matching decision operation, in which teeth cleaning matching decision information is transmitted from the dental cleaner terminal to the server.

3. The method of claim 1, further comprising performing a teeth cleaning guide information transmission operation, in which information of at least one of a teeth cleaning method or precautions when cleaning teeth is transmitted from the server to the dental cleaner terminal.

4. The method of claim 1, further comprising performing a before-teeth-cleaning dental condition information transmission operation, in which dental condition information including a teeth image of a user before teeth cleaning is transmitted from the dental cleaner terminal to the server.

5. The method of claim 4, further comprising performing an after-teeth-cleaning dental condition information transmission operation, in which dental condition information including a teeth image of the user after teeth cleaning is transmitted from the dental cleaner terminal to the server.

6. The method of claim 1, wherein the dental cleaner matching request operation is performed only when it is determined that user's agreement on protection of dental cleaner personal information is completed in the user terminal.

7. The method of claim 1, further comprising:
performing an image transmission operation, in which a body skeleton image of a user is transmitted from the user terminal to the server;
performing a skeleton condition analysis operation, in which skeleton information in which a skeleton balance state is digitized is derived by analyzing the body skeleton image in the server; and
performing a skeleton condition information transmission operation, in which the skeleton information is transmitted from the server to the user terminal.

8. The method of claim 7, further comprising performing a skeleton condition information usage agreement operation, in which information indicating user's agreement on usage of skeleton condition information of the user for a medical research purpose is transmitted from the user terminal to the server.

9. The method of claim 1,
wherein the method further uses a dentist terminal communicating with the server, and
wherein the method further comprises:
performing a dentist matching information transmission operation, in which correction service user information is transmitted from the user terminal to the server;
performing a dentist list transmission operation, in which a created dentist list is transmitted from the server to the user terminal; and
performing a correction treatment matching request operation, in which dentist information selected from the created dentist list is transmitted from the user terminal to the server.

10. The method of claim 9, further comprising performing a correction device delivery request operation, in which correction device delivery information is transmitted from the server to the dental cleaner terminal.
